(19) United States
(12) Patent Application Publication
(10) Pub. No.: US 2010/0249647 A1
(43) Pub. Date: Sep. 30, 2010

(54) Radiographic image capturing apparatus, biopsy apparatus, radiographic image capturing method, and biopsy method

(75) Inventor: Hiroki Nakayama, Kanagawa-ken (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/662,009

(22) Filed: Mar. 29, 2010

(30) Foreign Application Priority Data
Mar. 31, 2009 (JP) 2009-086245

(51) Int. Cl. A61B 10/02; A61B 6/02

(52) U.S. Cl. 600/567; 250/360.1

(57) ABSTRACT

A radiographic image capturing apparatus includes a radiation source for applying a radiation to an object to be examined, a radiation detector for detecting the radiation which has passed through the object and converting the detected radiation into a radiographic image, a positional information acquiring unit for acquiring positional information of the object, and an image capturing angle changer for changing an image capturing angle of the radiation source with respect to the radiation detector based on the positional information in a stereographic image capturing process for capturing at least two radiographic images of the object by applying the radiation to the object from directions which are different from each other.

### CROSS-REFERENCE TO RELATED APPLICATION

This application is based upon and claims the benefit of priority from Japanese Patent Application No. 2009-086245 filed on Mar. 31, 2009, of which the contents are incorporated herein by reference.

### BACKGROUND OF THE INVENTION

1. Field of the Invention

The present invention relates to a radiographic image capturing apparatus and a radiographic image capturing method for capturing at least two radiographic images of an object to be examined by irradiating the object with a radiation from directions which are different from each other.

The present invention is also concerned with a biopsy apparatus and a biopsy method for calculating the three-dimensional position of a region to be biopsied (hereinafter referred to as “biopsy region”) of the object based on the two radiographic images, moving a biopsy needle to the biopsy region based on the calculated three-dimensional position, and removing a tissue sample from the biopsy region with the biopsy needle.

2. Description of the Related Art

Heretofore, biopsy apparatus have been developed in the art for removing a tissue sample from a biopsy region of an object to be examined (e.g., an inflicted region of a breast of a subject) and examining the tissue sample for a disease diagnosis of the subject. Specifically, a biopsy apparatus is used in combination with a radiographic image capturing apparatus. The radiographic image capturing apparatus is used to capture a plurality of radiographic images by performing a stereographic image capturing process that applies a radiation to the object from directions which are different from each other. The biopsy apparatus specifies the three-dimensional position of the biopsy region based on the captured radiographic images. Then, the biopsy apparatus moves a biopsy needle to the biopsy region based on the specified three-dimensional position, and controls the biopsy needle to remove a tissue sample from the biopsy region.

According to Japanese Laid-Open Patent Publication No. 09-187447, it is proposed to change the distance between two radiation sources or the distance between two focal points of a single radiation source depending on the magnification ratio in a stereographic image capturing process.

When two radiographic images are captured in a stereographic image capturing process by irradiating an object to be examined with radiations emitted from radiation sources that are positioned to orient in two different directions, the positions (image capturing angles) of the radiation sources with respect to a radiation detector are fixed in advance.

An area irradiated with the radiation applied from the radiation source at one of the angles and an area irradiated with the radiation applied from the radiation source at the other angle are superposed on each other, and the superposed area serves as an area (examinable area) which is capable of specifying the three-dimensional position of the biopsy region. If the stereographic image capturing process is carried out while the biopsy region is placed out of the examinable area, then the object to be examined is exposed to unwanted radiations.

The examinable area is of such a configuration that it becomes progressively narrower in a direction from the radiation detector toward the radiation sources (see FIGS. 4A and 4B of the accompanying drawings). If the object thickness along the direction from the radiation detector toward the radiation source varies from object to object, then the image capturing angles may be fixed for an object to be examined which has a larger thickness. Once the image capturing angles have been fixed for the object to be examined which has the larger thickness, it is possible to specify the three-dimensional position of a biopsy region in that object to be examined. However, the thus-fixed examinable area for the object having the larger thickness may be too narrow for an object to be examined which has a smaller thickness, so that the biopsy region in the object having the smaller thickness may fall out of the examinable area.

### SUMMARY OF THE INVENTION

It is an object of the present invention to make it possible to change the image capturing angle of a radiation source in a stereographic image capturing process depending on an object to be examined and also to prevent the object to be examined from being exposed to unwanted radiations.

A radiographic image capturing apparatus according to the present invention comprises a radiation source for applying a radiation to an object to be examined, radiation detector for detecting the radiation which has passed through the object and converting the detected radiation into a radiographic image, a positional information acquiring unit for acquiring positional information of the object, and an image capturing angle changer for changing an image capturing angle of the radiation source with respect to the radiation detector based on the positional information in a stereographic image capturing process for capturing at least two radiographic images of the object by applying the radiation to the object from directions which are different from each other.

According to the present invention, there is also provided a biopsy apparatus for use with the above radiographic image capturing apparatus, the biopsy apparatus comprising a biopsy region positional information calculator for calculating a three-dimensional position of a biopsy region in the object based on the at least two radiographic images captured by the radiographic image capturing apparatus, and a biopsy needle for sampling a tissue from the biopsy region by piercing the biopsy region based on the three-dimensional position.

A radiographic image capturing method according to the present invention comprises the steps of acquiring positional information of an object to be examined with a positional information acquiring unit, changing an image capturing angle of a radiation source with respect to a radiation detector based on the positional information, with an image capturing angle changer, and performing a stereographic image capturing process for capturing at least two radiographic images of the object by applying a radiation to the object from the radiation source at changed image capturing angles which are different from each other.

A biopsy method according to the present invention comprises the steps of calculating a three-dimensional position of a biopsy region in the object based on the at least two radiographic images captured by the above radiographic image capturing method, with a biopsy region positional information calculator, and sampling a tissue from the biopsy region by piercing the biopsy region with a biopsy needle based on the three-dimensional position.

According to the present invention, as described above, the image capturing angle of the radiation source in the stereographic image capturing process is changed based on the positional information of the object to be examined. Therefore, the image capturing angle of the radiation source in the stereographic image capturing process can be changed based on the object to be examined, and the object to be examined is prevented from being exposed to unwanted radiations.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a perspective view of a radiographic image capturing apparatus according to an embodiment of the present invention;

FIG. 2 is an enlarged fragmentary side elevational view of the radiographic image capturing apparatus shown in FIG. 1;

FIG. 3 is a block diagram of the radiographic image capturing apparatus and a biopsy apparatus, according to the embodiment of the present invention;

FIGS. 4A and 4B are schematic views illustrative of a stereographic imaging process carried out by the radiographic image capturing apparatus;

FIG. 5 is a flowchart of an operation sequence of the radiographic image capturing apparatus and the biopsy apparatus according to the embodiment of the present invention;

FIGS. 6A through 6C are schematic views illustrative of a modified stereographic imaging process;

FIG. 7 is a flowchart of a modified operation sequence of the radiographic image capturing apparatus which carries out the modified stereographic imaging process illustrated in FIGS. 6A through 6C; and

FIG. 8 is a schematic view illustrative of an effect which a collimator has.

### DESCRIPTION OF THE PREFERRED EMBODIMENTS

A radiographic image capturing apparatus and a biopsy apparatus according to an embodiment of the present invention in relation to a radiographic image capturing method and a biopsy method carried thereby will be described below with reference to the drawings.

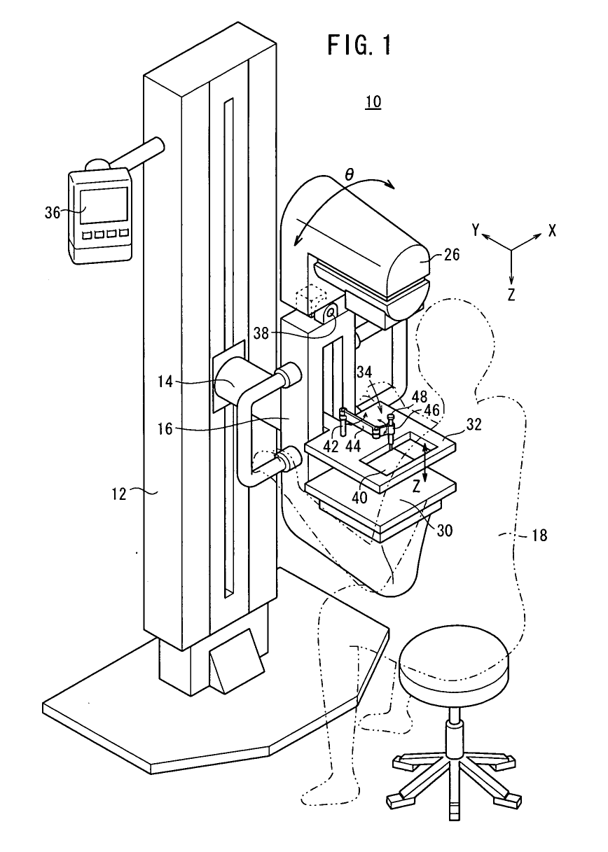

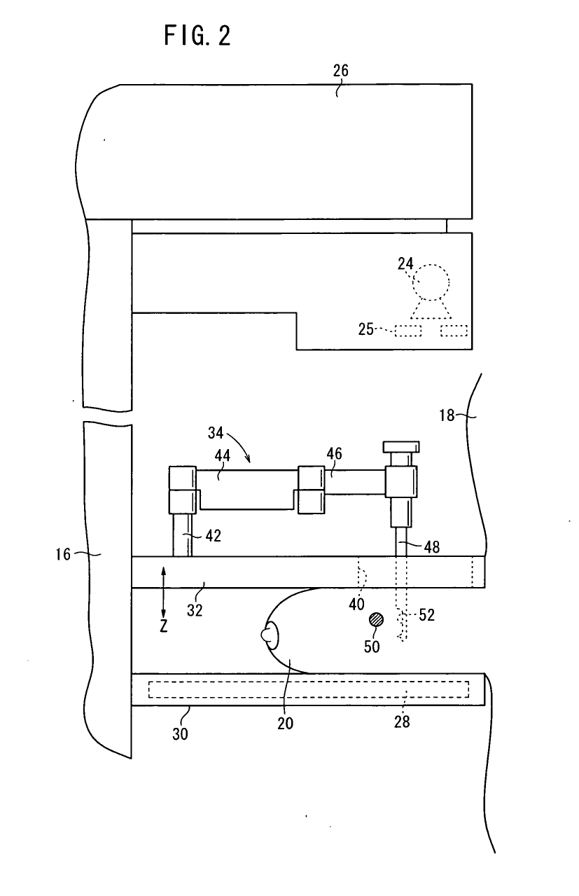

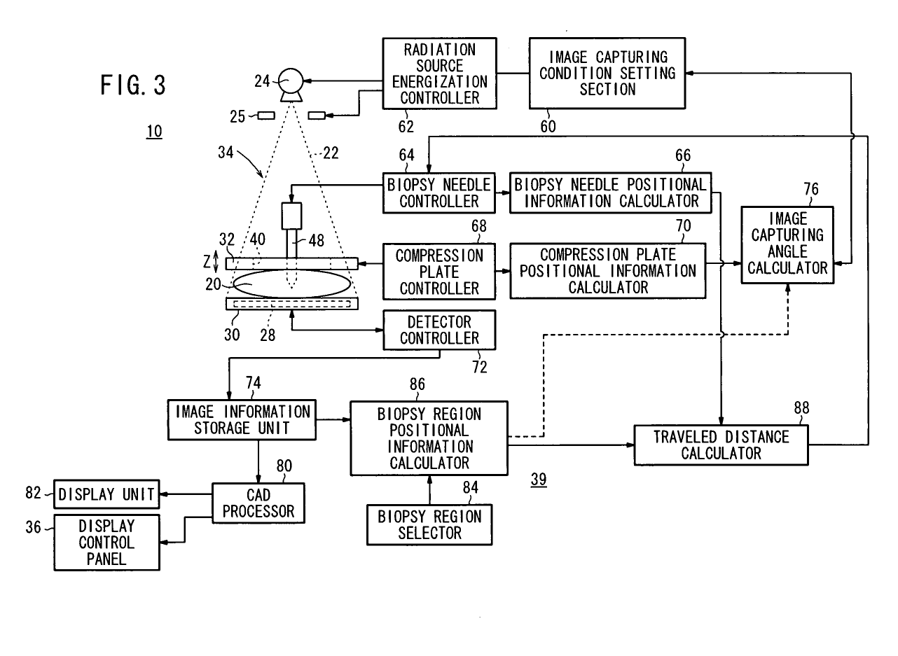

FIG. 1 shows a mammographic apparatus 10 serving as radiographic image capturing apparatus or a breast image capturing apparatus according to the embodiment of the present invention. As shown in FIG. 1, the mammographic apparatus 10 includes an upstanding base 12, a vertical arm 16 fixed to a horizontal swing shaft 14 disposed substantially centrally on the base 12, a radiation source housing unit 26 housing therein a radiation source 24 (see FIGS. 2 and 3) for applying radiation 22 (see FIG. 3) to a breast 20 (an object to be examined, see FIG. 3) of a subject 18, and which is fixed to an upper end of the arm 16, an image capturing base 30 mounted on a lower end of the arm 16 and housing therein a solid-state detector (radiation detector) 28 (see FIGS. 2 and 3) for detecting radiation 22 which has passed through the breast 20, a compression plate 32 for compressing and holding the breast 20 against the image capturing base 30, and a biopsy hand assembly 34 for removing a tissue sample from a biopsy region 50 of the breast 20, the biopsy hand assembly 34 being mounted on the compression plate 32. To the base 12, there is connected a display control panel 36 for displaying image capturing conditions representing an image capturing region, etc. of the subject 18, the ID information of the subject 18, etc., and setting these items of information, if necessary. The radiation source housing unit 26 also houses therein a collimator 25 for delimiting an irradiated field of the radiation 22 emitted from the radiation source 24.

When the arm 16, on which the radiation source housing unit 26 and the image capturing base 30 are secured, is angularly moved about the swing shaft 14, the direction of the radiation source housing unit 26 and the image capturing base 30 with respect to the breast 20 of the subject 18 is adjusted. The radiation source housing unit 26 is operatively coupled to the arm 16 by a hinge 38 and can be turned independently of the image capturing base 30 about the hinge 38 in the directions indicated by the arrow θ. The compression plate 32, which is coupled to the arm 16, is disposed between the radiation source housing unit 26 and the image capturing base 30. The compression plate 32 is vertically displaceable along the arm 16 in the directions indicated by the arrow Z.

The compression plate 32 has an opening 40 defined therein for allowing the biopsy hand assembly 34 to remove a tissue sample from the biopsy region 50 of the breast 20. The biopsy hand assembly 34 serves as part of a biopsy apparatus 39 (see FIG. 3) which is incorporated in the mammographic apparatus 10. The biopsy hand assembly 34 comprises a post 42 fixedly mounted on the compression plate 32, a first arm 44 having one end thereof pivotally supported on the post 42 so that the first arm 44 is angularly movable about the post 42 along the surface of the compression plate 32, and a second arm 46 having one end thereof pivotally supported on the other end of the first arm 44 so that the second arm 46 is angularly movable about the other end of the first arm 44 along the surface of the compression plate 32. A biopsy needle 48 is mounted on the other end of the second arm 46 for movement in the directions indicated by the arrow Z, which are perpendicular to the compression plate 32.

As shown in FIG. 2, the biopsy needle 48 has a sampler 52 near the lower end thereof for sampling, as a lesion location, a tissue (e.g., a calcified tissue) from the biopsy region 50 of the breast 20 under suction. The sampler 52 of the biopsy needle 48 can be moved to a position in the vicinity of the biopsy region 50 when the first arm 44 and the second arm 46 of the biopsy hand assembly 34 are moved in an X-Y plane parallel to the surface of the compression plate 32 and the biopsy needle 48 is moved in the directions indicated by the arrow Z.

FIG. 3 shows in block form a control circuit of the mammographic apparatus 10 including the biopsy apparatus 39.

As shown in FIG. 3, the mammographic apparatus 10 includes an image capturing condition setting section 60, a radiation source energization controller 62, a biopsy needle controller 64, a biopsy needle positional information calculator 66, a compression plate controller 68, a compression plate positional information calculator (positional information acquiring unit) 70, a detector controller 72, an image information storage unit 74, an image capturing angle calculator (image capturing angle changer) 76, a CAD (Computer Aided Diagnosis) processor 80, a display unit 82, a biopsy region selector 84, a biopsy region positional information calculator (positional information acquiring unit, positional information detector) 86, and a traveled distance calculator 88.

The biopsy needle controller 64, the biopsy needle positional information calculator 66, the biopsy region selector 84, the biopsy region positional information calculator 86, and the traveled distance calculator 88, as well as the biopsy hand assembly 34 and the biopsy needle tissue of the biopsy region 50, and are incorporated in the mammographic apparatus 10.

The image capturing condition setting section 60 sets image capturing conditions including a tube current and a tube voltage of the radiation source 24, types of a target and a filter that are set in the radiation source 24, an irradiation dose and an irradiation time of the radiation 22, the angle (image capturing angle) of the radiation source 24 with respect to the vertical axis (the directions indicated by the arrow Z) of the radiation detector 28 in a stereographic image capturing process, etc. The radiation source energization controller 62 controls the energization of the radiation source 24 according to the image capturing conditions. The biopsy needle controller 64 controls the biopsy hand assembly 34 (see FIGS. 1 and 2) to move the biopsy needle 48 to a desired position. The compression plate controller 68 moves the compression plate 32 in the directions indicated by the arrow Z. The detector controller 72 controls the solid-state detector 28 to store a radiographic image converted thereby into the image information storage unit 74.

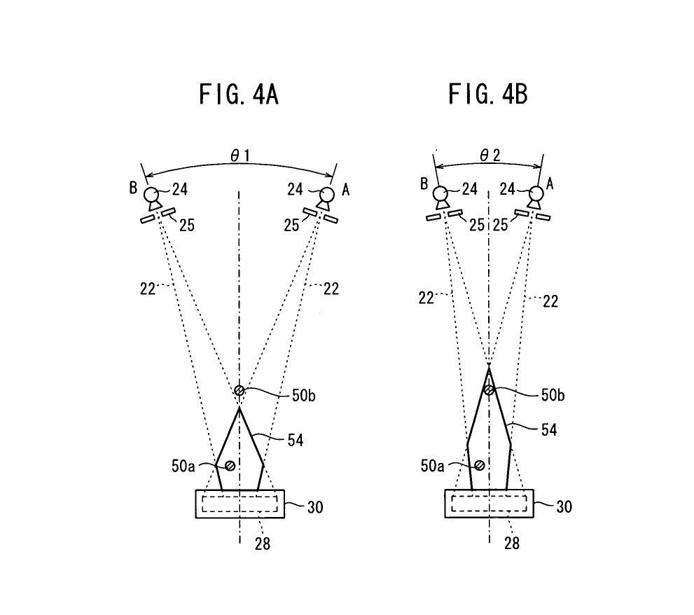

As shown in FIGS. 4A and 4B, the mammographic apparatus 10 is capable of performing a stereographic image capturing process for acquiring two radiographic images of the breast 20 in respective positions A, B by turning the radiation source housing unit 26 about the hinge 38 (see FIG. 1), placing the radiation source 24 in the positions A, B, and capturing radiographic images based on the radiation 22 emitted from the radiation source 24 which is placed in the positions A, B. In the stereographic image capturing process, specifically, the radiation source housing unit 26 is turned about the hinge 38 and the radiation source is placed in the positions A, B one at a time. Then, the radiation source 24 which is placed in the positions A, B applies the radiation 22 to the breast 20, and the radiation 22 passes the breast 20 to the solid-state detector 28, which converts the radiation 22 into radiographic images. The image information storage unit 74 now stores two radiographic images of the breast 20 at the respective positions A, B.

The CAD processor 80 processes the two radiographic images stored in the image information storage unit 74 and displays the processed radiographic images on the display unit 82 and the display control panel 36.

The biopsy region selector 84 comprises a pointing device such as a mouse or the like. The doctor or radiological technician in charge who has seen the displayed contents, i.e., the two radiographic images, on the display unit 82 and/or the display control panel 36 can select one, from which a tissue is to be removed, of a plurality of biopsy regions 50 in the displayed two radiographic images, using the pointing device as the biopsy region selector 84. Specifically, the doctor or radiological technician selects a biopsy region 50 in one of the two radiographic images and also selects a corresponding biopsy region 50 in the other of the two radiographic images.

The biopsy region positional information calculator 86 calculates the three-dimensional position of the selected biopsy region 50 based on the positions of the selected biopsy region 50 in the two radiographic images. The three-dimensional position of the selected biopsy region 50 can be calculated according to a known three-dimensional position calculating scheme for the stereographic image capturing process.

The biopsy needle positional information calculator 66 calculates the positional information of the tip end of the biopsy needle 48 which has been moved by the biopsy needle controller 64. The traveled distance calculator 88 calculates the distance by which the biopsy needle 48 is to move with respect to the biopsy region 50, based on the three-dimensional position of the biopsy region 50 which has been calculated by the biopsy region positional information calculator 86 and the position of the tip end of the biopsy needle 48 which has been calculated by the biopsy needle positional information calculator 66. Based on the calculated distance by which the biopsy needle 48 is to be moved with respect to the biopsy region 50, the biopsy needle controller 64 moves the biopsy needle 48 for removing a tissue sample from the selected biopsy region 50.

The compression plate positional information calculator 70 calculates the positional information of the compression plate 32 which has been moved with respect to the image capturing base 30 by the compression plate controller 68. Since the compression plate 32 presses the breast 20 with respect to the image capturing base 30 and holds the breast 20 in the pressed state, the positional information of the compression plate 32 represents the thickness information of the breast 20 in the pressed state.

As shown in FIGS. 4A and 4B, a space where areas irradiated with the radiations applied from the radiation source 24 in the positions A, B are superposed on each other, serves as an examinable area 54 which is capable of specifying the three-dimensional position of the biopsy region 50.

In FIGS. 4A and 4B, there are two biopsy regions 50a, 50b that are present in the breast 20 which is relatively thick, the breast 20 being omitted from illustration.

FIG. 4A shows a situation wherein the angle θ1 formed between the position A and the position B is relatively large, i.e., the image capturing angle θ1/2 is relatively large, and the biopsy region 50a closer to the solid-state detector 28 is positioned within the examinable area 54 while the biopsy region 50b closer to the radiation source 24 is positioned out of the examinable area 54. The image capturing angle θ1/2 in FIG. 4A is preset in the image capturing condition setting section 60. Even if the stereographic image capturing process is carried out with the image capturing angle θ1/2, the biopsy region positional information calculator 86 is unable to calculate the three-dimensional position of the biopsy region 50b.

Thus, prior to the stereographic image capturing process, the image capturing angle calculator 76 calculates an image capturing angle θ2/2 (θ1>θ2) with which the examinable area 54 extends to the surface of the pressed breast 20 which faces the radiation source 24, based on the positional information of the compression plate 32 which is representative of the thickness information of the pressed breast 20, and outputs the calculated image capturing angle θ2/2 to the image capturing condition setting section 60, which changes the preset image capturing angle θ1/2 to the calculated image capturing angle θ2/2. In other words, as the breast 20 is thicker, the image capturing angle calculator 76 sets the image capturing angle to a smaller value, i.e., makes smaller the angle formed between the position A and the position B, and as the breast 20 is thinner, the image capturing angle calculator 76 sets the image capturing angle to a larger value, i.e., makes larger the angle formed between the position A and the position B. If the stereographic image capturing process is carried out with the image capturing angle θ2/2, then the biopsy region positional information calculator 86 is able to calculate the three-dimensional position of the biopsy region 50b.

The image capturing angle calculator 76 compares the calculated image capturing angle and the image capturing angle preset in the image capturing condition setting section 60 with each other. If the image capturing angle preset in the image capturing condition setting section 60 is an angle depending on the thickness of the breast 20, then the image capturing angle calculator 76 does not change the image capturing angle preset in the image capturing condition setting section 60.

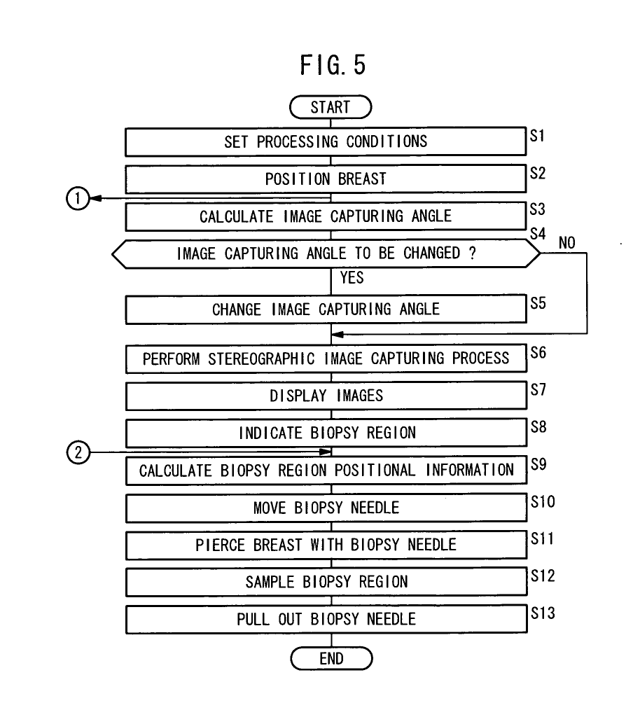

The mammographic apparatus 10 according to the embodiment of the present invention is basically constructed as described above. Operation of the mammographic apparatus 10 will be described below with reference to a flowchart shown in FIG. 5.

In step S1 shown in FIG. 5, the image capturing condition setting section 60 sets image capturing conditions including a tube current and a tube voltage of the radiation source 24, an irradiation dose and an irradiation time of the radiation 22, an image capturing method, an image capturing angle of the radiation source 24, etc. The set image capturing conditions are set in the radiation source energization controller 62.

Then, the radiological technician positions the breast 20 of the subject 18 according to the indicated image capturing method in step S2. Specifically, the radiological technician places the breast 20 in a predetermined position on the image capturing base 30, and then energizes the compression plate controller 68 to move the compression plate 32 toward the image capturing base 30 in the downward direction indicated by the arrow Z, thereby compressing and positioning the breast 20 against the image capturing base 30.

In step S3, the compression plate positional information calculator 70 calculates the positional information of the compression plate 32 with respect to the image capturing base 30, and outputs the calculated positional information to the image capturing angle calculator 76. The image capturing angle calculator 76 calculates an image capturing angle depending on the thickness of the pressed breast 20 based on the positional information of the compression plate 32.

In step S4, the image capturing angle calculator 76 compares the calculated image capturing angle and the image capturing angle preset in the image capturing condition setting section 60 with each other to determine whether or not the preset image capturing angle is an angle depending on the thickness of the breast 20. If the image capturing angle preset in the image capturing condition setting section 60 is not an angle depending on the thickness of the breast 20, but is of such an angle that the biopsy region 50b is positioned out of the examinable area 54 (“YES” in step S4), then the image capturing angle calculator 76 outputs the calculated image capturing angle to the image capturing condition setting section 60. Thus, the image capturing angle preset in the image capturing condition setting section 60 is changed to the calculated image capturing angle in step S5.

If the image capturing angle preset in the image capturing condition setting section 60 is an angle depending on the thickness of the breast 20 (“NO” in step S4), then the image capturing angle calculator 76 skips the processing of step S5.

After the above process sequence for the image capturing angle has been finished, the mammographic apparatus 10 energizes the radiation source 24 to perform a stereographic image capturing process on the breast 20 in step S6. Specifically, the radiation source housing unit 26 is turned about the hinge 38 (see FIG. 1) to place the radiation source 24 successively in the position A and the position B (see FIG. 4B). When the radiation source 24 is placed successively in the position A and the position B, it emits respectively the radiations 22 which pass through the breast 20 and are applied to the solid-state detector 28 in the image capturing base 30. The solid-state detector 28 now detects radiographic images of the breast 20 based on the radiations 22 emitted from the radiation source 24 placed in the positions A, B and transmitted through the breast 20. The detector controller 72 controls the solid-state detector 28 to acquire the radiographic images of the breast 20 based on the radiations 22 from the radiation source 24 in the positions A, B, and stores the acquired two radiographic images into the image information storage unit 74.

In step S7, the CAD processor 80 processes the'two radiographic images stored in the image information storage unit 74 and displays the processed radiographic images on the display unit 82 and the display control panel 36.

In step S8, the doctor or the radiological technician selects one, from which a tissue is to be removed, of a plurality of biopsy regions 50 in the two radiographic images displayed on the display unit 82 and/or the display control panel 36, using the biopsy region selector 84 which is a pointing device such as a mouse.

When the desired biopsy region 50 is selected with the biopsy region selector 84, the biopsy region positional information calculator 86 calculates the three-dimensional position of the selected biopsy region 50 based on the position of the selected biopsy region 50 in the two radiographic images in step S9. The traveled distance calculator 88 calculates the distance by which the biopsy needle 48 is to move with respect to the biopsy region 50, based on the three-dimensional position of the biopsy region 50 which has been calculated by the biopsy region positional information calculator 86 and the position of the tip end of the biopsy needle 48 which has been calculated by the biopsy needle positional information calculator 66.

In step S10, based on the calculated distance from the traveled distance calculator 88, the biopsy needle controller 64 moves the biopsy needle 48 for sampling a tissue from the biopsy region 50. The biopsy hand assembly 34 moves the first arm 44 and the second arm 46 in the X-Y plane to position the biopsy needle 48 above the biopsy region 50. Then, the biopsy hand assembly 34 moves the biopsy needle 48 in the downward direction indicated by the arrow Z, and inserts the biopsy needle 48 into the breast 20 through the opening 40 in the compression plate 32 in step S11.

When the sampler 52 of the biopsy needle 48 reaches a position near the biopsy region 50, the biopsy needle 48 starts to sample a tissue from the biopsy region 50 under suction in step S12. Thereafter, the biopsy needle controller 64 moves the biopsy needle 48 in the upward direction indicated by the arrow Z until the biopsy needle 48 is pulled out of the breast 20 in step S13. The tissue sampling process is now finished.

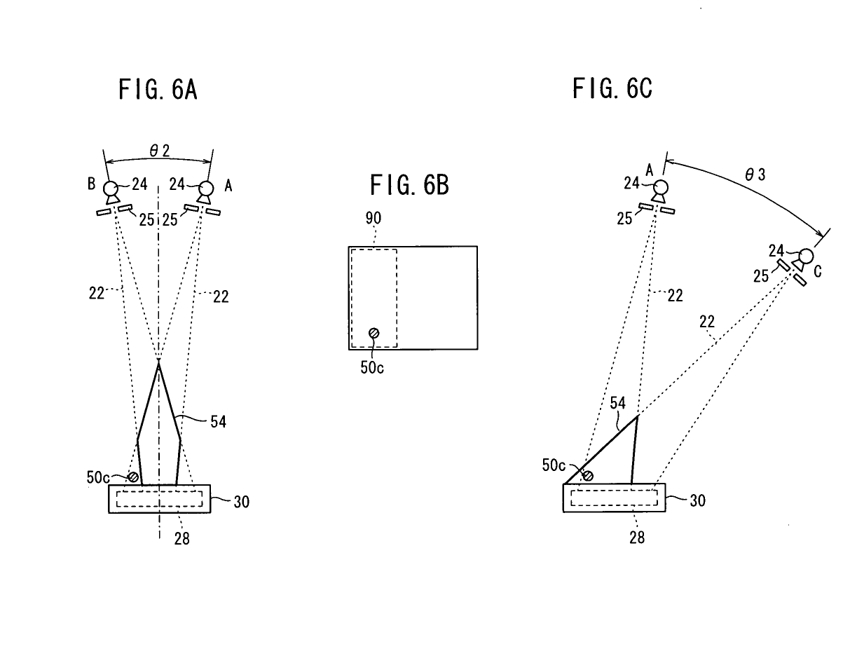

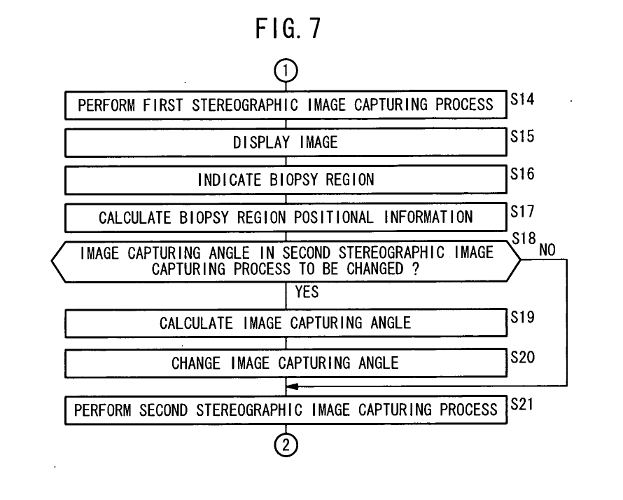

The mammographic apparatus 10 according to the above embodiment operates as described above. Steps S3 though S8 shown in FIG. 5 may be modified according to a modification shown in FIGS. 6A through 6C and 7.

According to the modification, a first image capturing process is carried out based on the image capturing conditions set in the image capturing condition setting section 60, and the image capturing angle of the radiation source 24 in a second image capturing process is changed based on a first radiographic image produced by the first image capturing process.

FIG. 6A illustrates a stereographic image capturing process carried out based on the image capturing conditions set in the image capturing condition setting section 60. In FIG. 6A, a biopsy region 50c in the breast 20 is positioned out of the examinable area 54. Even if the stereographic image capturing process is carried out according to the given image capturing conditions, the biopsy region positional information calculator 86 is unable to calculate the three-dimensional position of the biopsy region 50c.

According to the modification, based on the position of the biopsy region 50c in a first radiographic image (FIG. 6B) produced by the first image capturing process of the stereographic image capturing process, the image capturing angle calculator 76 calculates an image capturing angle (an image capturing angle at a position C in FIG. 6C) for a second image capturing process. The image capturing angle calculator 76 sets the calculated image capturing angle in the image capturing condition setting section 60, after which the second image capturing process is carried out. Specifically, the image capturing angle calculator 76 calculates an image capturing angle at the position C which is opposite to the position B across the position A, as an image capturing angle for a second image capturing process, so that the biopsy region 50c will be included in the examinable area 54. The reference numeral 90 in FIG. 6B denotes an image of the examinable area 54 in FIG. 6C projected onto the radiographic image. In FIG. 6C, θ3 represents the angle formed between the position A and the position C.

FIG. 7 is a flowchart of a modified operation sequence of the radiographic image capturing apparatus which carries out the modified stereographic imaging process. In step S14 after step S2 shown in FIG. 5, the mammographic apparatus 10 energizes the radiation source 24 to perform a first image capturing process of the stereographic image capturing process on the breast 20. The first image capturing process is carried out at the image capturing angle at the position A, for example (see FIG. 6C). The solid-state detector 28 detects the radiation 22 which has passed through the breast 20 to capture a first radiographic image of the breast 20 at the position A, and the detector controller 72 stores the first radiographic image of the breast 20 at the position A into the image information storage unit 74.

In step S15, the CAD processor 80 processes the first radiographic image stored in the image information storage unit 74 and displays the processed first radiographic image on the display unit 82 and the display control panel 36.

In step S16, the doctor or the radiological technician selects a biopsy region 50c, from which a tissue is to be removed, in the first radiographic image displayed on the display unit 82 and/or the display control panel 36, using the biopsy region selector 84.

In step S17, the biopsy region positional information calculator 86 calculates the position of the biopsy region 50c selected using the biopsy region selector 84. At this time, the biopsy region positional information calculator 86 calculates the two-dimensional position of the biopsy region 50c in the first radiographic image.

In step S18, the image capturing angle calculator 76 calculates a present examinable area 54 based on the image capturing angle set in the image capturing condition setting section 60, and determines whether the biopsy region 50c is positioned out of the examinable area 54 or not, i.e., whether the image capturing angle in a second image capturing process is to be changed or not.

If the image capturing angle set in the image capturing condition setting section 60 is an image capturing angle that places the biopsy region 50c out of the examinable area 54 (“YES” in step S18), then the image capturing angle calculator 76 calculates an image capturing angle that depends on the thickness of the breast 20 and that places the biopsy region 50c within the examinable area 54, based on the two-dimensional position of the biopsy region 50c in the first radiographic image and the thickness information of the breast 20 in step S19. The image capturing angle calculator 76 outputs the calculated image capturing angle to the image capturing condition setting section 60, which changes the preset image capturing angle to the calculated image capturing angle in step S20.

If the image capturing angle set in the image capturing condition setting section 60 is an image capturing angle that places the biopsy region 50c within the examinable area 54 (“NO” in step S18), then the processing of steps S19, 20 is skipped.

After the above-mentioned operation sequence, the mammographic apparatus 10 energizes the radiation source 24 to perform a second image capturing process of the stereographic image capturing process on the breast 20 in step S21. In the second image capturing process, the radiation source 24 is placed in the position C and applies the radiation 22 to the breast 20. The solid-state detector 28 of the image-capturing base 30 detects the radiation 22 which has passed through the breast 20. The detector controller 72 controls the solid-state detector 28 to acquire a second radiographic image of the breast 20 at the position C, and stores the second radiographic image of the breast 20 at the position C into the image information storage unit 74.

The biopsy region positional information calculator 86 is now able to calculate the three-dimensional position of the biopsy region 50c in step S9 shown in FIG. 5. The mammographic apparatus 10 then performs the processing from step S10 shown in FIG. 5.

According to the present embodiment, as described above, the image capturing angle of the radiation source 24 in the stereographic image capturing process is changed based on the thickness information of the breast 20. Therefore, the image capturing angle of the radiation source 24 in the stereographic image capturing process can be changed based on the thickness of the breast 20, and the breast 20 is prevented from being exposed to unwanted radiations.

Since the image capturing angle calculator 76 makes smaller an image capturing angle for the radiation source 24 at the positions A, B as the thickness of the breast 20 is larger, the three-dimensional position of a biopsy region 50 in the breast 20 of large thickness can reliably be calculated.

According to the modification, the biopsy region positional information calculator 86 calculates the position of the biopsy region 50c in the first radiographic image captured in the first image capturing process of the stereographic image capturing process. If the biopsy region 50c is positioned out of the examinable area 54, then the image capturing angle calculator 76 changes the image capturing angle in the second first image capturing process based on the calculated position of the biopsy region 50c. Consequently, an unwanted stereographic image capturing process is reliably prevented from being carried out.

According to the present embodiment, the mammographic apparatus 10 includes the collimator 25 for delimiting an irradiated field of the radiation 22 emitted from the radiation source 24. The collimator 25 controls the irradiated field to fall within a certain range depending on the image capturing angle.

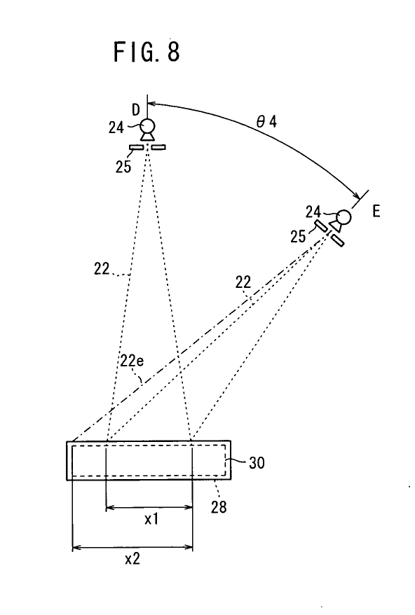

More specifically, as shown in FIG. 8, when a stereographic image capturing process is carried out at an image capturing angle for the radiation source 24 at positions D, E, an irradiated field of the radiation 22 from the position D is indicated by x1 and an irradiated field of the radiation 22 from the position E is indicated by x2. If an angle θ4 of the position E with respect to the position D is large, then the irradiated field of the radiation 22 from the position E may become too wide so that a radiation 22e applied onto the left side of the irradiated field x1 does not contribute to the calculation of the three-dimensional position of the biopsy region 50.

According to the present embodiment, the aperture of the collimator 25, which is defined by a plurality of shutter plates of the collimator 25, is changed in shape to reduce the irradiated field of the radiation 22 from the position E into alignment with the irradiated field x1. In this manner, the stereographic image capturing process and the calculation of the three-dimensional position of the biopsy region 50 are carried out efficiently, and the breast 20 is prevented from being exposed to unwanted radiations.

Although certain preferred embodiments of the present invention have been shown and described in detail, it should be understood that various changes and modifications may be made therein without departing from the scope of the appended claims.

What is claimed is:

1. A radiographic image capturing apparatus comprising:
a radiation source for applying a radiation to an object to be examined;
a radiation detector for detecting the radiation which has passed through the object and converting the detected radiation into a radiographic image;
a positional information acquiring unit for acquiring positional information of the object; and
an image capturing angle changer for changing an image capturing angle of the radiation source with respect to the radiation detector based on the positional information in a stereographic image capturing process for capturing at least two radiographic images of the object by applying the radiation to the object from directions which are different from each other.

2. A radiographic image capturing apparatus according to claim 1, wherein the object is a breast of a subject, and the radiographic image capturing apparatus comprises a breast image capturing apparatus comprising:
an image capturing base for holding the breast thereon, the image capturing base housing the radiation detector therein; and
a compression plate displaceable toward the image capturing base for compressing the breast against the image capturing base;
wherein the positional information acquiring unit comprises a positional information calculator for calculating a thickness of the breast along a direction in which the breast is compressed, based on the position of the compression plate; and
the image capturing angle changer changes the image capturing angle based on the calculated thickness of the breast.

3. A radiographic image capturing apparatus according to claim 1, wherein the image capturing angle changer makes smaller an image capturing angle formed by the radiation source positioned in the directions which are different from each other, as the object is positionally nearer the radiation source.

4. A radiographic image capturing apparatus according to claim 1, further comprising:
a collimator for controlling an irradiated field of the radiation;
wherein the collimator controls the irradiated field to fall within a range depending on the image capturing angle.

5. A radiographic image capturing apparatus according to claim 1, wherein the positional information acquiring unit comprises a positional information detector for detecting the position of the object based on a first one of the radiographic images captured by the stereographic image capturing process; and
the image capturing angle changer changes the image capturing angle of the radiation source for capturing a second one of the radiographic images captured by the stereographic image capturing process, based on the position of the object detected by the positional information detector.

6. A biopsy apparatus for use with a radiographic image capturing apparatus including a radiation source for applying a radiation to an object to be examined, a radiation detector for detecting the radiation which has passed through the object and converting the detected radiation into a radiographic image, a positional information acquiring unit for acquiring positional information of the object, and an image capturing angle changer for changing an image capturing angle of the radiation source with respect to the radiation detector based on the positional information in a stereographic image capturing process for capturing at least two radiographic images of the object by applying the radiation to the object from directions which are different from each other, the biopsy apparatus comprising:
a biopsy region positional information calculator for calculating a three-dimensional position of a biopsy region in the object based on the at least two radiographic images; and
a biopsy needle for sampling a tissue from the biopsy region by piercing the biopsy region based on the three-dimensional position.

7. A radiographic image capturing method comprising the steps of:
acquiring positional information of an object to be examined with a positional information acquiring unit;
changing an image capturing angle of a radiation source with respect to a radiation detector based on the positional information, with an image capturing angle changer; and
performing a stereographic image capturing process for capturing at least two radiographic images of the object by applying a radiation to the object from the radiation source at changed image capturing angles which are different from each other.

8. A biopsy method comprising the steps of:
acquiring positional information of an object to be examined with a positional information acquiring unit;
changing an image capturing angle of a radiation source with respect to a radiation detector based on the positional information, with an image capturing angle changer;
performing a stereographic image capturing process for capturing at least two radiographic images of the object by applying a radiation to the object from the radiation source at changed image capturing angles which are different from each other;
calculating a three-dimensional position of a biopsy region in the object based on the at least two radiographic images, with a biopsy region positional information calculator; and
sampling a tissue from the biopsy region by piercing the biopsy region with a biopsy needle based on the three-dimensional position.
